(19) United States
(12) Patent Application Publication
(10) Pub. No.: US 2007/0026410 A1
(43) Pub. Date: Feb. 1, 2007

(54) Nucleoside cyanine dye derivatives for RNA labeling and nucleic acid detection and methods for using same

(76) Inventors: C.J. Yu, Pasadena, CA (US); Faqing Huang, Hattiesburg, MS (US); Xiaoping Ying, Pasadena, CA (US)

Correspondence Address: Adegenix, 870 S. Myrtle Ave., Monrovia, CA, 91016, US

(21) Appl. No.: 11/192,505

(22) Filed: Jul. 29, 2005

(51) Int. Cl. C12Q 1/68; C07H 19/16; C07H 21/04

(52) U.S. Cl. 435/6; 536/27.3; 536/25.32

(57) ABSTRACT

Fluorescent adenosine derivatives that can be incorporated into the 5′ end of transcribed RNA are disclosed. Adenosine derivatives have readily detectable cyanine dyes attached. Symmetric derivatives are disclosed that. The transcribed RNAs can be used in a variety of applications including nucleic acid detection and in the study of RNA structure and function. Methods of generating labeled RNAs from a biological sample also are disclosed.

### FIELD OF THE INVENTION

The invention relates to adenosine derivatives labeled with cyanine dyes at 5′ end, and methods and materials for their preparation and use.

### BACKGROUND

The labeling of nucleic acids is essential for a variety of molecular biology techniques as well as diagnostic assays. Many techniques for labeling involve non-isotopic labeling strategies, which are advantageous because of the stability of the label, the lack of radioactivity and the versatility of detection in a variety of formats. Labeled RNA molecules, for example, are useful for microarray analysis of gene expression and for genotyping applications.

Recently, a new technology has been invented for initiating transcription of RNA with adenosine derivatives. The techniques are described in publications: F. Huang, Efficient incorporation of CoA, NAD and FAD into RNA by in vitro transcription. Nucl. Acids. Research, 2003, Vol. 31, No. 3, e8 and F. Huang et al., Synthesis of adenosine derivatives as transcription initiators and preparation of 5′ fluorescein- and biotin-labeled RNA though one-step in vitro transcription. RNA, 2003, 9: 1562-1570 and in U.S. Pat. Appl'n. 20040241649. According to the techniques described in the Huang publications, a template having an RNA polymerase promoter sequence is transcribed in vitro in the presence of an adenosine derivative.

PNK-catalyzed phosphorylation by [γ-³²P]-ATP is a common procedure to prepare 5′ ³²P-labeled RNA that is required in various studies of structure, function and mechanism. However, such a ³²P-based RNA labeling method may present a series of problems for the experimenters. First, ³²P radioactivity quickly diminishes with time due to its short half-life. In addition, RNA labeling efficiency by [γ-³²P]-ATP actually decreases much faster than the radiodecay of ³²p, probably due to ATP damage caused by strong 32P radioactivity. Therefore, usable [γ-³²P]-ATP has an even shorter half-life (<14 days). Frequent supply of fresh [γ-³²P]-ATP is necessary for efficient 5′ RNA labeling by ³²P and PNK. Unless it is used by several experimenters in a sizable laboratory or shared among different laboratories, a substantial amount of [γ-³²P]-ATP is wasted due to its fast radiodecay. Second, 5′ ³²P-labeled RNA may slowly loose its function during storage as a result of structural damage by its 32P radioactivity. Accordingly, it may be necessary to frequently prepare freshly 32P-labeled RNA before a previous preparation is fully consumed. Third, some RNA may present difficulties for efficient labeling by PNK and [γ-³²P]-ATP. Lastly, ³²P is a source of radio-hazard. Its use requires user training and strict workplace safety regulation.

Cyanine dyes are well known and have been used for a variety of biological applications. Particularly useful cyanine dyes include those disclosed in U.S. Pat. No. 5,268,486 and the dyes CYDYES, CY3 and CY5 (Amersham, Inc.). Cyanine dyes have been used to label DNA oligonucleotides by incorporation using standard phosphoramidite chemistry (U.S. Pat. Nos. 5,556,959 and 5,808,044).

The fluorescent compositions described herein are useful for the 5′-labeling of nucleic acids and address some of the limitations of radioactive and other approaches for generating labeled RNA. The invention also provides methods for generating nucleic acid fragments.

### SUMMARY

The invention provides fluorescently labeled adenosine derivatives that are useful for the 5′-labeling of RNA by in vitro transcription using an RNA polymerase system. The adenosine derivatives of the invention have a 5′-linkage to a fluorescent cyanine dye moiety.

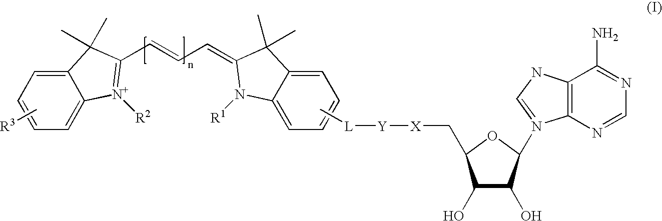

A compound of the invention is shown in structure I. R¹, R², and R³ are independently selected from optionally substituted alkyl, alkenyl, aryl, and alkylaryl groups. R¹, R^{2,} and R³ can be optionally substituted with one or more heteroatoms such as O, S, N, etc or other groups, including charged groups such as amino groups, carboxylate groups, and the like. In one embodiment, R¹ and R² are the same. R¹ and R² are preferably lower alkyl groups of C₁ to C₆ and ether linkages with six to eight atoms. In a preferred embodiment, R³is —CH₂COOH.

X is —CH₂, —CO—, —O—, —S—, —NH—, —NR—, —ONH—, or —CONH—. Y is —CH₂—, —CO—, —PO₃—, P(S)O₂—, —SO₂—, —COO—, —CO—, —PO₂NH, S or O. Together X and Y form any number of covalent linkages including, for example, alkyl, amide, disulfide, ether, ester, carbamate, phosphodiester, phoshorothioate, sulfoxide, and thioether.

L is a linker of at least about 2 and up to about 100 atoms in length. The linker is preferably an alkyl chain that can be optionally substituted with one or more heteroatoms or other groups. The chain can include, for example, one or more ether, amide, ester, carbamate, disulfide or other linkages. The linker can be composed of a number of units linked through such linkages. In one embodiment, the linker is: —CH₂—CO—NH—(CH₂)₆—NH.

The linkage to the dye can be made through any suitable position on the dye. In a preferred embodiment, the linkage is made as shown in structure II.

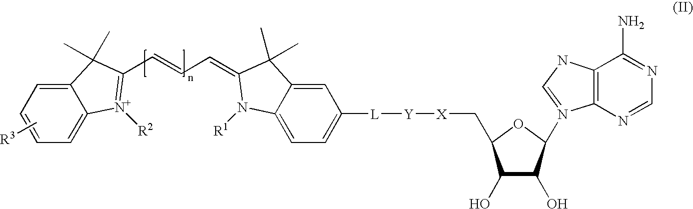

The compounds of the invention generally are salts, with appropriate counterions. For example, where X and Y form a phospodiester or phosphoramide group, the phosphate will generally have a positively charged counterion such as Na⁺, Li^{+,} K⁺ or NH₄⁺.

The invention includes a second class of labeled adenosine derivatives, having two adenosine molecules linked to a single cyanine dye molecule through a 5′ linkage to each adenosine moiety. These molecules can be synthesized readily and avoid the need for asymmetric protection strategies. Also, di-substituted derivatives will be incorporated into RNA biopolymers at a higher rate than will singly labeled adenosine derivatives. Therefore, the concentration of the dye-labeled adenosine can be decreased by up to about 50% compared to use of the singly labeled adenosine. The molecules of this second class include compounds with the structure:

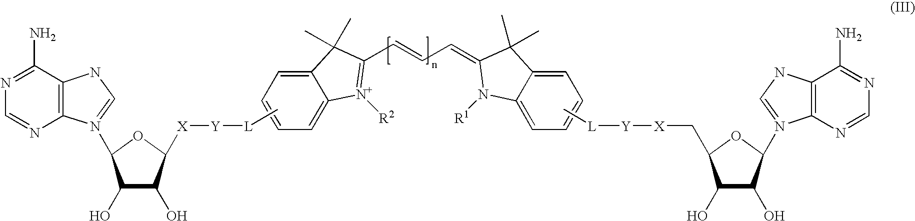

R¹, R² are independently selected from optionally substituted alkyl, alkenyl, alkylaryl. R¹, R² can be optionally substituted with one or more heteroatoms or other groups, including charged groups such as amino groups, carboxylate groups, and the like. In one embodiment, R¹ and R² are the same. R¹ and R² are preferably lower alkyl groups of C₁ to C₄ and alkyl ethers six to eight atoms. In a preferred embodiment, R³ is —CH₂COOH.

X is —CH₂, —CO—, —O—, —S—, —NH—, —NR—, —ONH—, or —CONH—. Y is —CH₂—, —CO—, —PO₃—, P(S)O₂—, —SO₂—, —COO—, —CO—, —PO₂NH, S or O. Together X and Y form acovalent linkages including, for example, alkyl, amide, disulfide, ether, ester, carbamate, phosphodiester, phoshorothioate, sulfoxide, and thioether linkages.

L is a linker of at least about 2 and up to about 100 atoms in length. The linker is preferably an alkyl chain that can be optionally substituted with one or more heteroatoms or other groups. The chain can include, for example, one or more ether, amide, ester, carbamate, disulfide or other linkages. The linker can be composed of a number of units linked through such linkages. In one embodiment, the linker is: —CH₂—CO—NH—(CH₂)₆—NH.

R¹ and R² are an optionally substituted alkyl, alkenyl, aryl, or alkylaryl group.

In one embodiment, linkage to the dye is made as shown in structure IV:

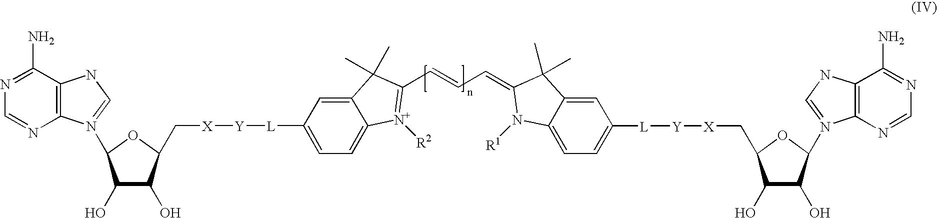

The invention also provides methods for generating labeled RNA probes from a biological sample. According to the methods of the invention, a biological sample containing a nucleic acid molecule is amplified at a locus using a primer having an RNA polymerase primer sequence therein. The locus is amplified using a technique such as PCR to create an amplicon having the locus sequence of the DNA along with the primer sequence. An oligonucleotide complementary to the promoter sequence can be added to the reaction mixture. A labeled RNA molecule is then produced by transcribing the amplicon with an appropriate RNA polymerase in the presence of a labeled initiator nucleoside, preferably an adenosine derivative. The labeled nucleoside can be fluorescently labeled or can be labeled with an electrochemical tag, a metal complex, an affinity tag such as biotin, a co-enzyme or other tag. In a preferred embodiment, the labeled nucleoside is an adenosine derivative of the invention.

The methods of the invention are useful for generating one or more labeled RNA molecules. According to the methods of the invention, a plurality of primers can used, having the same promoter sequence as one another, or having different promoter sequences. When the sequences are the same, the transcription reaction can be carried out to produce a plurality of distinct RNA molecule simultaneously. The labeled RNA molecules that are produced can be used for a variety of purposed, including, for example, detection of sequences within a sample, use with microarray experiments, or for blotting or other experiments that require a labeled probe.

### BRIEF DESCRIPTION OF THE FIGURES

FIG. 1 shows a symmetric di-adenosine derivative of the invention.

FIG. 2 shows the synthesis of compounds 3 and 4.

FIG. 3 shows a synthetic pathway to symmetrically labeled di-adenosine derivatives 14 (F550/570) and 15 (F650/670).

FIG. 4 shows a synthetic pathway to mono-substituted derivatives of the invention.

FIG. 5 shows absorption and emission spectra of compounds 14 (F550/570) and 15 (F650/670).

FIG. 6 shows a gel of RNA labeled with compounds 14 (F550/570) and 15 (F650/670).

FIG. 7 shows a scheme for generating RNA molecules from a biological sample containing DNA.

### DETAILED DESCRIPTION

Before the present invention is described in detail, it is to be understood that this invention is not limited to the particular complexes, methods or articles described, as such complexes, methods or articles can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise.

Definitions

“Alkyl” refers to a branched, unbranched or cyclic saturated hydrocarbon group of 1 to 100 carbon atoms optionally substituted at one or more positions. Examples of alkyl groups include methyl, ethyl, n-propyl, isopropyl, n-butyl, s-butyl, t-butyl, n-amyl, isoamyl, n-hexyl, n-heptyl, n-octyl, n-decyl, hexyloctyl, tetradecyl, hexadecyl, eicosyl, tetracosyl and the like, as well as cycloalkyl groups such as cyclopropyl, cyclobutyl, cyclopentyl, cyclohexyl, cycloheptyl, cyclooctyl and the like. The term “lower alkyl” refers to an alkyl group of 1 to 6 carbon atoms, preferably 1 to 4 carbon atoms. Exemplary substituents on substituted alkyl groups include hydroxyl, cyano, alkoxy, ═O, ═S, NO₂, halogen, haloalkyl, heteroalkyl, amine, thioether and —SH.

“Alkoxy” refers to an alkyl group having one or more carbons substituted with oxygen atoms.

“Alkenyl” refers to a branched, unbranched or cyclic hydrocarbon group of 2 to 24 carbon atoms containing at least one carbon-carbon double bond optionally substituted at one or more positions. Examples of alkenyl groups include ethenyl, 1-propenyl, 2-propenyl (allyl), 1-methylvinyl, cyclopropenyl, 1-butenyl, 2-butenyl, isobutenyl, 1,4-butadienyl, cyclobutenyl, 1-methylbut-2-enyl, 2-methylbut-2-en-4-yl, prenyl, pent-1-enyl, pent-3-enyl, 1,1-dimethylallyl cyclopentenyl, hex-2-enyl, 1-methyl-1-ethylallyl, cyclohexenyl, heptenyl, cycloheptenyl, octenyl, cyclooctenyl, decenyl, tetradecenyl, hexadecenyl, eicosenyl, tetracosenyl and the like. Preferred alkenyl groups herein contain 2 to 12 carbon atoms.

“Amine” refers to an —N(R′)R″ group, where R′ and R″ are independently selected from hydrogen, alkyl, aryl, and alkylaryl.

“Aryl” refers to an aromatic group which has at least one ring having a conjugated pi electron system and includes carbocyclic, heterocyclic and polycyclic aryl groups, and can be optionally substituted at one or more positions. Typical aryl groups contain 1 to 5 aromatic rings, which may be fused and/or linked. Exemplary aryl groups include phenyl, furanyl, azolyl, thiofuranyl, pyridyl, pyrimidyl, pyrazinyl, triazinyl, indenyl, benzofuranyl, indolyl, naphthyl, quinolinyl, isoquinolinyl, quinazolinyl, pyridopyridinyl, pyrrolopyridinyl, purinyl, tetralinyl and the like. Exemplary substituents on optionally substituted aryl groups include alkyl, alkoxy, alkylcarboxy, alkenyl, alkenyloxy, alkenylcarboxy, aryl, aryloxy, alkylaryl, alkylaryloxy, fused saturated or unsaturated optionally substituted rings, halogen, haloalkyl, heteroalkyl, —S(O)R, sulfonyl, —SO.sub.3 R, —SR, —NO.sub.2, —NRR′, —OH, —CN, —C(O)R, —OC(O)R, —NHC(O)R, —(CH2).sub.n CO.sub.2 R or —(CH2).sub.n CONRR′ where n is 0-4, and wherein R and R′ are independently H, alkyl, aryl or alkylaryl.

“Halo” or “halogen” refers to fluoro, chloro, bromo or iodo, and usually relates to halo substitution for a hydrogen atom in an organic compound.

“Heteroalkyl” refers to an alkyl group wherein one or more carbon atoms and associated hydrogen atom(s) are replaced by an optionally substituted heteroatom, and includes alkyl groups substituted with only one type of heteroatom as well as alkyl groups substituted with a mixture of different types of heteroatoms. Heteroatoms include oxygen, sulfur, and nitrogen. As used herein, nitrogen heteroatoms and sulfur heteroatoms include any oxidized form of nitrogen and sulfur, and any form of nitrogen having four covalent bonds including protonated forms. An optionally substituted heteroatom refers to replacement of one or more hydrogens attached to a nitrogen atom with alkyl, aryl, alkylaryl or hydroxyl.

“Optional” or “optionally” means that the subsequently described event or circumstance may or may not occur, and that the description includes instances where said event or circumstance occurs singly or multiply and instances where it does not occur at all. For example, the phrase “optionally substituted alkylene” means an alkylene moiety that may or may not be substituted and the description includes both unsubstituted, monosubstituted, and polysubstituted alkylenes. An optionally substituted alkyl group also include the omission of Hydrogen atoms, to form alkenyl or alkynyl groups. Optionally substituted alkyl groups also include alkyl groups having one or more carbon atoms replaced with a heteroatom that itself can be substituted or not. For example, an alkyl ether is an optionally substituted alkyl group. Likewise, a “heteroatom-inserted alkyl group” is has one or more carbon atoms replaced with a heteroatom that itself can be substituted or not. Such heteroatom-inserted alkyl groups include, for example, ethers, thioethers and the like.

A “substituent” refers to a group that replaces one or more hydrogens attached to a carbon or nitrogen. Exemplary substituents include alkyl, alkylidenyl, alkylcarboxy, alkoxy, alkenyl, alkenylcarboxy, alkenyloxy, aryl, aryloxy, alkylaryl, alkylaryloxy, —OH, amide, carboxamide, carboxy, sulfonyl, .═O, ═S, —NO₂, halogen, haloalkyl, fused saturated or unsaturated optionally substituted rings, —S(O)R, —SO₃ R, —SR, —NRR′, —OH, —CN, —C(O)R, —OC(O)R, —NHC(O)R, —(CH₂)ₙCO₂R or —(CH2)ₙCONRR′ where n is 0-4, and wherein R and R′ are independently H, alkyl, aryl or alkylaryl. Substituents also include replacement of a carbon atom and one or more associated hydrogen atoms with an optionally substituted heteroatom.

Synthesis of Compounds.

The adenosine derivatives of the invention can be synthesized by one of skill in the art of organic chemistry, following in part the teachings of U.S. Pat. Nos. 5,268,486 5,556,959 and 5,808,044, as well as Mujumdar et al. Cytometry 10: 11-19 (1989); Ernst et al. Cytometry 10: 3-10 (1989); Mujumdar et al. Bioconjugate Chemistry 4 105-111; and Southwick et al. Cytometry 11:418-430 (1990). Particular synthetic schemes for the derivatives are shown in Schemes III, IV and V.

In vitro Transcription.

The compounds of the invention are useful for initiating transcription of RNA molecules in vitro. The molecules can be used to transcribe single or double-stranded templates in the presence of a polymerase that can initiate transcription using an adenosine derivative. The polymerase is generally one suitable for in vitro tracription reactions, for example the well defined T3, T7, and SP6 polymerases. In a preferred embodiment, the polymerase is a T7 polymerase and the promoter is a T7 class II promoter.

In transcription where an adenosine derivative (R-A) other than ATP is present, both normal RNA (pppRNA) and adenosine derivative-linked RNA (R-A-RNA) are produced. Although high concentrations of adenosine derivatives lead to high relative yields of R-A-RNA over total RNA (pppRNA versus R-A-RNA), total RNA yields may decrease at high concentrations of adenosine derivatives. Importantly, the symmetric di-adenosine derivatives of the invention can be used at lower concentrations than mono-labeled derivatives.

To balance the total RNA yields and relative yields of adenosine derivative-linked RNA, transcription in the presence of 1 mM each of the four nucleoside triphosphates and about 1 to 4 mM of an adenosine derivative can be performed. However, if higher relative yields of adenosine derivative-linked RNA are desired and lower total RNA yields are acceptable, higher concentrations of adenosine derivatives or lower concentrations of ATP may be used. At higher than 8 mM of adenosine derivative concentrations, total RNA yields can be significantly lower. Optimization of transcription conditions for a particular adenosine derivative, promoter and template can be useful for improving yields.

The promoter sequence used will be selected in conjunction with the polymerase that is to be used for the transcription reaction. The polymerase system selected must be one that can use adenosine to initiate transcription. The preferred promoter sequence is that of the T7 Class II promoter (5 ′-TAA TAC GAC TCA CTA TTA GGA G-3′; SEQ ID NO. 1).

Generally, the transcription reaction will contain ATP, UTP, GTP and CTP. In some cases one of the bases can be omitted. Derivatives of the naturally-occurring bases also can be used. Analogs also can be used, including those with reactive groups that can be used to attach, for example, secondary labels. The analogs also themselves can be labeled. When an analog or derivative is used, the analog or derivative can be used in place of the naturally occurring nucleotide or can be used in combination with the naturally occurring nucleotide. The relative concentrations of the analog or derivative to the naturally occurring nucleotide can be selected to vary the amount of incorporation of the analog or derivative relative to the naturally occurring nucleotide.

The reaction mixture can further comprise buffers, surfactants, salts, RNAse inhibitors and other common molecular biology reagents. The reaction mixture is selected to be suitable for RNA transcription. Generally the reaction conditions are optimized in terms of the choice of buffer, pH, time and temperature according to the activity requirements of the RNA polymerase. When the enzyme is commercially available, conditions recommended by the manufacturer are typically used. For example, T7 RNA polymerase can be used with a solution of 40 mM Tris (pH 8.0), 6 mM MgCl₂, 2 mM spermidine, 0.01% Triton X-100, 5 mM DTT, and 0.2 units/uL RNAse inhibitor. Incubation is typically performed for about one hour to about four hours at a temperature of about 37° C., or according to the manufacturer's protocols, or protocols developed that result in transcription.

Nucleic Acid Molecules

One embodiment of the invention includes RNA molecules containing adenosine derivatives covalently attached-to the 5′ end of the RNA molecule. The RNA molecule can generally have any RNA sequence. The length of the RNA sequence can generally be any number of bases. For example, the RNA sequence can be at least about 5 bases, at least about 10 bases, at least about 20 bases, at least about 30 bases, at least about 40 bases, at least about 50 bases, at least about 60 bases, at least about 70 bases, at least about 80 bases, at least about 90 bases, at least about 100 bases, at least about 200 bases, at least about 300 bases, at least about 400 bases, at least about 500 bases, at least about 600 bases, at least about 700 bases, at least about 800 bases, at least about 900 bases, at least about 1000 bases, at least about 2000 bases, and so on. There is no practical upper limit to the length of the RNA sequence, as it is dependent primarily upon the length of the template nucleic acid sequence used in its preparation (or upon the synthetic method used). The RNA molecule can be purified, isolated, or can be present as a mixture of RNA molecules.

Kits

An additional embodiment of the invention is related to kits useful for the preparation of the above described RNA molecules. The kits can comprise reagents, enzymes, nucleic acid templates, one or more containers, buffers, solvents, instruction protocols, purification materials, positive and negative controls, standards, and so on.

An example of such a kit can comprise a RNA polymerase enzyme, one or more of the previously discussed adenosine derivatives, ATP, UTP, GTP, and CTP.

Methods of Preparation of RNA Molecules

Another aspect of the invention relates to methods of preparing RNA molecules having adenosine derivatives of the invention at their 5′ terminal end. Generally, the method comprises providing a DNA template comprising a RNA polymerase promoter sequence; contacting the DNA template, an RNA polymerase enzyme, an adenosine derivative, ATP, UTP, GTP, and CTP to prepare a reaction mixture; and incubating the reaction mixture under conditions suitable for RNA transcription to prepare RNA molecules. It is presently preferred that the method is an in vitro enzymatic method. The DNA template can generally be any DNA template. The DNA template can be single stranded or double stranded. The length of the DNA sequence can generally be any number of bases or base pairs. For example, the DNA sequence can be at least about 5 bases, at least about 10 bases, at least about 20 bases, at least about 30 bases, at least about 40 bases, at least about 50 bases, at least about 60 bases, at least about 70 bases, at least about 80 bases, at least about 90 bases, at least about 100 bases, at least about 200 bases, at least about 300 bases, at least about 400 bases, at least about 500 bases, at least about 600 bases, at least about 700 bases, at least about 800 bases, at least about 900 bases, at least about 1000 bases, at least about 2000 bases, and so on. There is no practical upper limit to the length of the DNA sequence. The DNA sequence can be a single DNA sequence, or a mixture of DNA sequences.

It is presently preferred that the promoter sequence be the T7 Class II promoter sequence and the RNA polymerase enzyme is the T7 RNA polymerase enzyme.

The adenosine derivative can be any adenosine derivative or coenzyme discussed above. The reaction mixtures typically will contain ATP, UTP, GTP, and CTP in order to produce a transcribed RNA molecule containing A, U, G, and C. It is possible that some DNA templates may lack one or more of the four bases. In such a case, one or more of ATP, UTP, GTP, and CTP could be omitted. For example, if the DNA template to be transcribed contained only A, G, and C (i.e. no T), then UTP could be omitted from the reaction mixture as the transcribed RNA would contain only A, G, and C.

In order to prepare a radiolabeled RNA molecule, the reaction mixture can further comprise radioactive components such as α-³²P-ATP, α-³²P-GTP, α-³²P-CTP, α-³²p-UTP, α-³⁵S-ATP, α-³⁵S-GTP, α-³⁵S-CTP, and α-³⁵S-UTP.

The method can further comprise one or more purification steps after the incubation step. The purification step can involve use of gel electrophoresis (such as polyacrylamide gel electrophoresis; PAGE), membrane filtration, or liquid chromatography. The method can further comprise visualizing and/or quantifying the prepared RNA molecules using fluorescence, phosphorimaging, or other radioimaging methods.

### EXAMPLES

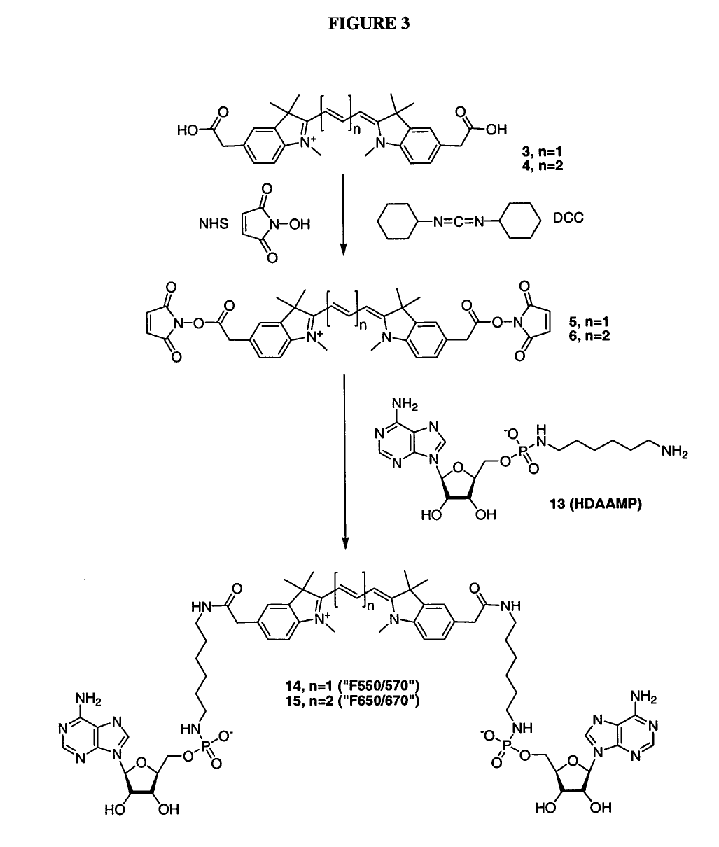

FIGS. 2 and 3 shows the synthesis of mono- and di-substituted derivatives of adenosine. A symmetric cyanine dye (preferably with n=1 or n=2) having two carboxylic acids was activated to form the N-hydroxy succinimidyl ester by treatment with NHS and dicyclocarbodiimide (DCC). The di-activated dye compound was reacted with the primary amine derivative 5′HDA-AMP to give a mixture of mono- and di-substituted adenosine derivatives. The derivatives can be separated, for example, by reversed-phase HPLC to give pure mono- and di-substituted adenosine derivatives. For some applications, a mixture of the derivatives also can be used.

Synthesis of Cyanine-AMP Conjugates

All reagents and chemicals were purchased from Aldrich and used as received. Synthetic procedures are shown in FIGS. 2 and 3. Starting from 2,3,3-trimethyl-3H-indole-5-acetic acid 1 (Southwick, P. L., Carins, J. G., Ernst, L. A., Waggoner, A. S. and Alan, S. (1988) Org. Prep. Proced. Int., 20, 279-284), the common intermediate 1,2,3,3-tetramethyl-indoleninium-5-acetate (2) was synthesized by methylation of 1. Condensation of two molecules of 2 with one molecule of triethyl orthoformate (Southwick, P. L., Ernst, L. A., Tauriello, E. W., Parker, S. R., Mujumdar, R. B., Mujumdar, S. R., Clever, H. A., Waggoner, A. S. and Alan, S. (1990) Cytometry, 11, 418-430) afforded the symmetrical red cyanine dye 3 with two free carboxyl groups that can be used for subsequent conjugation with AMP via a linker. Separately, Condensation of one molecule of 1,3,3-triemethoxypropene with two molecules of 2 produced another symmetrical blue cyanine dye 4 with two free carboxyl groups. The carboxyl groups of 3 and 4 were then activated by N,N′-dicyclohexylcarbodiimide (DCC) to form their corresponding hydroxysuccinimide (NHS) esters, 5 and 6. Finally, the intermediates 5 and 6 were individually coupled to 5′-(6-aminohexyl) adenosine phosphoramidate (HDAAMP) (19) to afford a pair of novel symmetrical cyanine-AMP conjugates, 14, F550/570, and 15, F650/670. Detailed description of the syntheses of 2-6, F550/570, and F650/670 are as following:

Synthesis of compound 2: To a 25 mL-Shlenk tube was added 0.5 g (2.3 mmol) of compound 1, prepared from the published procedure (Southwick et al., 1988), 1.2 mL (19.3 mmol) of iodomethane and 10 mL of acetonitrile. The reaction mixture was degassed with argon for 30 min and the tube was sealed with a cap and heated in an oil bath at 80° C. for 1 h. After cooling, the reaction mixture was transferred into a flask and concentrated under vacuum to give 0.78 g (94.4 %) of product 2.

Synthesis of compound 3: The literature procedures (Southwick et al., 1988, 1990) were used to prepare compound 3. To 1.0 g (2.8 mmol) of compound 2 was added 20 mL of dry pyridine. While the reaction mixture was refluxing, 1.39 mL (8.4 mmol) of triethyl orthoformate was added slowly (0.4 mL per 15 min). After completion of addition, the reaction mixture was refluxed for another 2 h. Solvent was removed and the red residue was dissolved in 40 mL of methanol, followed by adding 200 mL of ethyl acetate. After concentrating to about 50 mL, another 100 mL of ethyl acetate was added, and concentrated to about 50 mL. Into this suspension was added 100 mL of ethyl acetate, the top solvent was decanted and the red residue was dried over under vacuum to give 0.81 g (96.4 %) of the compound 3. MS analysis gave the following results: C₂₉H₃₃N₂O₄⁺, calcd., 473.24, found 473.2 (M⁺).

Synthesis of compound 4: The literature procedure (21) was used to prepare compound 4. To 1.0 g (2.8 mmol) of compound 2 was added 28 mL of dry acetonitrile, 0.6 mL of triethylamine (TEA) and 0.2 mL of acetic acid. While the reaction mixture was refluxing, a solution of 1.0 g (7.6 mmol) of 1,3,3-trimethoxypropene in 4.0 mL of acetonitrile was added slowly (0.5 mL per 15 min). After completion of addition, the reaction mixture was refluxed for another 2 h. Solvent was removed and the blue/purple residue was dissolved in 40 mL of methanol, followed by adding 200 mL of ethyl acetate. After concentrating to about 50 mL, another 100 mL of ethyl acetate was added, and concentrated to about 50 mL. Into this suspension was added another 100 mL of ethyl acetate, the top solvent was decanted and the red reside was dried over a high vacuum to give 0.84 g (96.5 %) of the compound 4. The molecular peak found by MS, 499.2 (M⁺), is consistent with the expected formula C₃₁H₃₅N₂O₄⁺, 499.26.

Synthesis of 5 and 6: To 100 mg (0.16 mmol) of compound 3 or 4 was added 90 mL of CH₂Cl₂, 100 mg (0.48 mmol) of N,N′-dicyclohexylcarbodiimide (DCC), 55 mg (0.48 mmol) of N-hydroxysuccinimide (NHS) and 50 mg of 4-(dimethylamino)pyridine (DMAP). The reaction mixture was stirred for 2 h. The urea derivative precipitate was formed and filtered off. The filtrate was concentrated to dryness and used for the next step of coupling reaction without further purification. MS analysis gave the following results: 5-C₃₇H₃₉N₄O₈⁺, calcd., 667.28, found 667.2 (M⁺); 6-C₃₉H₄₁N₄O₈⁺, calcd., 693.29, found 693.2 (M⁺).

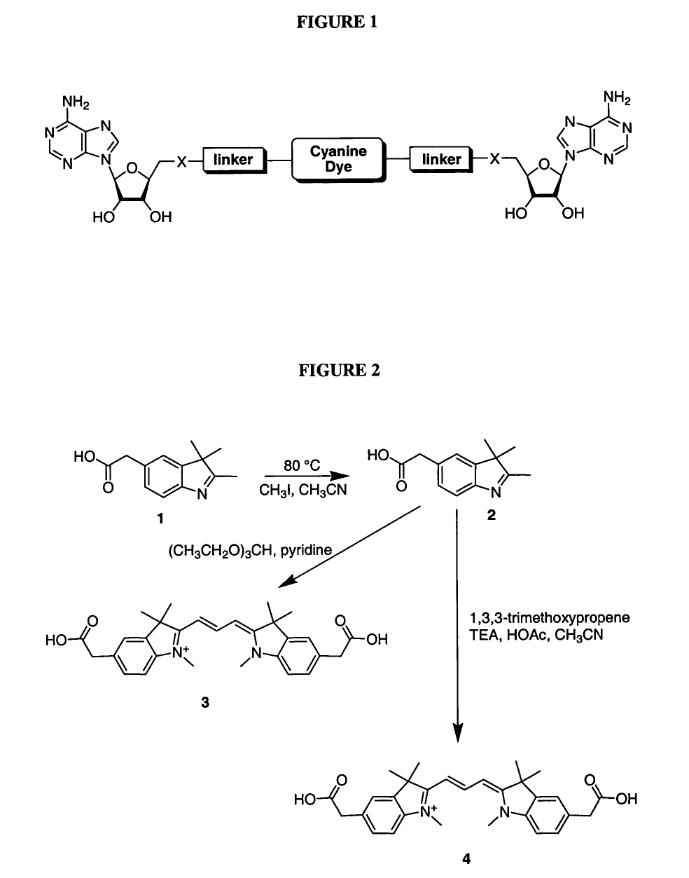

Synthesis and purification of F550/570 and F6501670: To a 300 μL aqueous solution of 370 mM HDAAMP (19) was added 150 μL TEA, 750 μL N,N-dimethylformide (DMF) and 20 mg of 5 or 6, which was dissolved separately in 150 μL DMF. After 30 min of reaction, 1.2 mL of water was added to the sample. The resulting solution was filtered with a 0.2 μm syringe filter. Purification of F550/570 and F650/670 was achieved by semi-preparative reverse phase HPLC (FIG. 1 & 2). Isolated yields for F550/570 and F650/670 were ˜20%. High resolution MS analysis gave excellent results: F550/570 -C₆₁H₈₅N₁₆O₁₄P₂⁺, Calcd., 1327.5901, found 1327.5869 (M⁺); F650/670 -C₆₃H₈₇N₁₆O₁₄P₂⁺, Calcd., 1353.6057, found 1353.5998 (M⁺).

Spectroscopic Properties of F550/570 and F650/670

UV absorbance spectra and molar extinction coefficiencies of F550/570 and F650/670 were obtained from a JASCO spectrometer (V-530) in 20 mM phosphate, pH 7.0. Fluorescence emission spectra were measured with an ISS PC1 Fluorometer (Champaign, Ill.) in 20 mM phosphate, pH 7.0, under the excitation of 510 nm and 610 nm for F550/570 and F650/670, respectively.

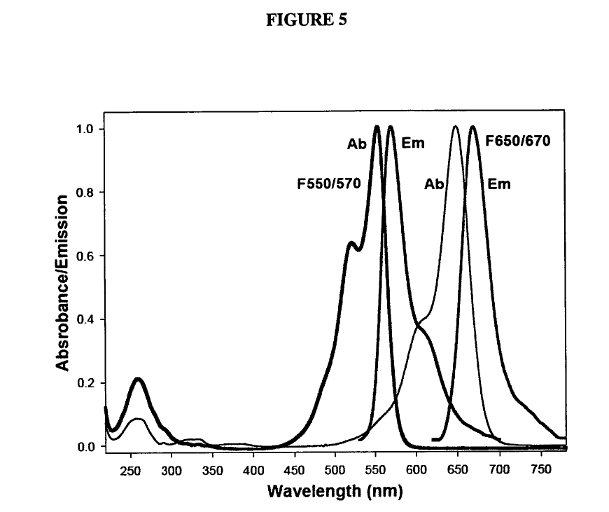

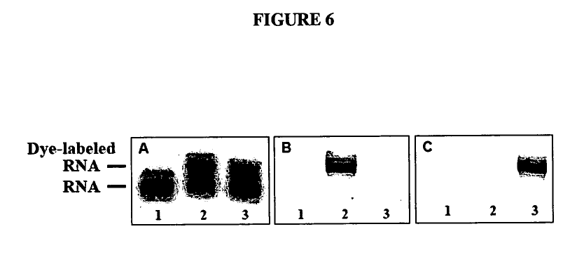

Ultraviolet and visible spectra of F550/570 and F650/670 (FIG. 5, Ex lines) show additive contribution from both the adenosines and the cyanine cores. For both cyanine-AMP conjugates, the absorbance within 220-300 nm is contributed mainly by the adenosine moieties. The absorbance between 450-580 nm of F550/570, with λₘₐₓ at 550 nm, is purely due to the cyanine dye 3 core. Similarly, for F650/670, the absorbance between 580-700 nm (λₘₐₓ=650 nm) is originated from the cyanine dye 4 core. Molar extinction coefficiencies measured in 20 mM phosphate buffer, pH 7.0, are ε₅₅₀=˜130,000 M⁻¹.cm⁻¹ and ε₆₅₀=˜210,000 M⁻¹.cm⁻¹ for F550/570 and F650/670, respectively. Fluorescence emission spectra of F550/570 and F650/670 are marked as Em curves in FIG. 5. F550/570 fluoresces between 550-700 nm, with emission λₘₐₓ=570 nm. Under excitation, F650/670 emits fluorescence within 640-780 nm (λₘₐₓ=670 nm). Within the visible range (440-780 nm), both the absorption spectra and fluorescence emission spectra of F550/570 and F650/670 are similar to those of common cyanine dyes, Cy3 and Cy5, respectively (23). The attachment of two adenosines and HDA linkers at the opposite ends of the cyanine cores has no apparent effects on spectroscopic properties of the cyanine dye within the visible range.

RNA 5′ labeling by F550/570 and F650/670

Fluorescent labeling of 5′ RNA by F550/570 and F650/670 was performed under normal in vitro transcription conditions (18,19) with slight modifications: changing [ATP] from 1 mM to 0. 25 mM and adding 2 mM of F550/570 or F650/670 to the transcription solution. The final transcription solution contained 40 mM Tris.HCl, pH 8.0, 5 mM DTT, 6 mM MgCl₂, 2 mM spermidine, 0.01% Triton X-100, 0.25 mM ATP, 1 mM each of UTP, GTP, and CTP, 2 mM F550/570 or F650/670, 0.05-0.5 μM dsDNA containing the T7 φ2.5 promoter (18,19), 500 units of T7 RNA polymerase per 100 μL reaction, 10-20 units of RNase inhibitor per 100 μL reaction. Also included was 2 μL of [α-³²P]-ATP per 100 μL reaction as a tracer for RNA analysis. The labeling reaction was carried out at 37° C. for 2 hours before analysis by denaturing PAGE. Product detection and quantitation were achieved by phosphorimaging under the excitation by ³²P, a 532 nm green laser, and a 633 nm red laser (Typhoon 9400, Amersham Biosciences).

Fluorescent Labeling of 5′ RNA by F550/570 and F650/670

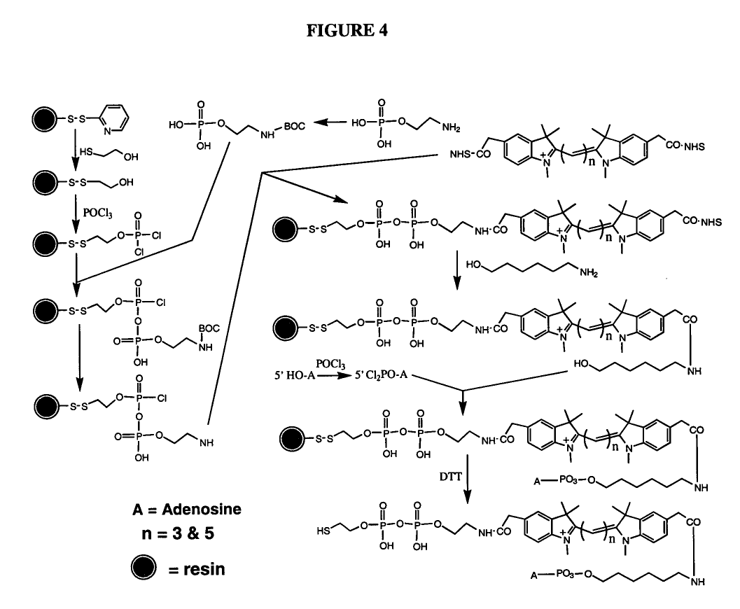

Fluorescent labeling of 5′ RNA is achieved by simply including F550/570 or F650/670 in transcription solutions under the T7 class II promoter φ02.5 (18,19). One of the two adenosines within F550/570 or F650/670 initiates transcription, resulting in 5′ RNA labeling by the cyanine dyes. Although there are two identical adenosines within F550/570 or F650/670, the probability of both adenosines initiating transcription to produce head-to-head joined RNA via F550/570 or F650/670 is low due to the high concentration ratios of F550/570 (or F650/670) over transcribed RNA molecules (i.e., mM vs μM). Because neither F550/570 nor F650/670 contains a nucleoside 5′-triphosphate, the cyanine dyes cannot be incorporated into internal RNA positions by T7 RNA polymerase. FIG. 4 shows 5′ RNA labeling by F550/570 and F650/670. Three parallel transcription experiments (with [α-³²P]-ATP as the internal radiolabel) were carried out in the absence of the cyanine dyes (lane 1) or in the presence of F550/570 (lane 2) or F650/670 (lane 3). Phosphorimaging based on 32p (FIG. 4A) revealed an additional slower RNA band in lane 2 and lane 3. Scanning of the same gel under the excitation with the 532 nm green laser (FIG. 4B) shows only a single RNA band in lane 2, whose location overlaps with that of the upper band of lane 2 in FIG. 4A. Under the excitation of a 633 nm red laser (FIG. 4C), scanning of the same gel displays another single RNA band in lane 3, whose location superimposes with that of the upper band of lane 3 in FIG. 4A. Taken together, the three different scannings of the same gel based on excitation by ³²p (FIG. 4A), 532 nm photons (FIG. 4B), and 633 nm photons (FIG. 4C) indicate fluorescent labeling of RNA by F550/570 (lane 2) and F650/670 (lane 3) during transcription. The labeling yields are 60% and 35% of total RNA for F550/570 and F650/670, respectively. In different experiments with varying RNA sequences, RNA labeling yields by F550/570 were consistently high (50-70%), and 30-50% RNA labeling yields were achieved by F650/670.

Since no transcription-based methods would produce 100% labeled RNA, isolation of labeled RNA from unlabeled (normal) RNA may be required for its applications. Due to their relatively large sizes, F550/570- and F650/670-labeled RNA displays significant migration retardation (5-6 nt difference) by PAGE. This added property of F550/570- and F650/670-labeled RNA may be exploited to achieve high purity levels of fluorescent RNA by PAGE. In one experiment, we have purified an F550/570-labeled 120-nt RNA to >95% purity using 8% denaturing PAGE (35×40×0.04 cm).

Synthesis of Mono-substituted Derivatives

FIG. 4 shows a synthetic scheme for constructing a mono-substituted cyanine dye derivative. Activated disulfide resin is reacted with an alkyl hydroxyl thiol. The free alcohol is phosphorylated with POCl₃, and reacted with a protected alkyl amino phosphate to give resin linked diphosphate. Upon deprotection of the amino protecting group, the resin is treated with the di-NHS ester of a cyanine dye. The resin-linked dye complex is treated with an alkyl amino alcohol which reacts at the remaining NHS ester. The resin-linked alcohol is then treated with activated adenosine monophosphoryl dichloride. The complex can be removed from the resin by reduction of the disulfide bond, for example, with dithiothreitol (DTT).

Labeling RNA from Biological Samples

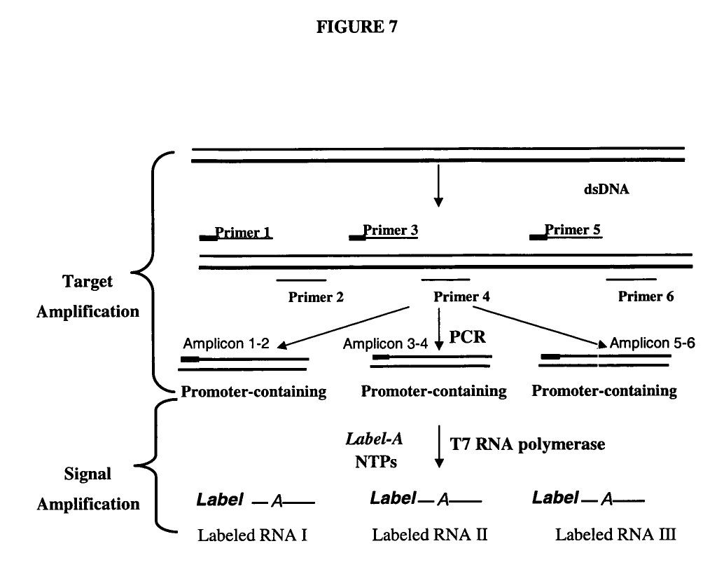

As shown in FIG. 7, RNA from one of more loci can be produced according to the methods of the invention. Primers 1, 3 and 5 contain a promoter (schematically shown as the bold region). Upon amplification by PCR, using primers 2, 4, and 6, amplicons 1-2, 3-4, and 5-6 are produced. The amplicons have promoter sequences corresponding to those of the primers 1, 3 and 5. The primers can have the same or different sequences. Amplicons 1-2, 3-4, and 5-6 are then transcribed in the presence of an adenosine derivative, including those of the invention having a cyanine dye. The resulting labeled RNA produced (Labeled RNA I, II, and III) has the label. The RNA could be used for example to genotype a biological sample at a number of loci simultaneously.

We claim:

1. An adenosine derivative having a cyanine dye linked to the 5′ end.

2. The adenosine derivative of claim 1, having the structure: 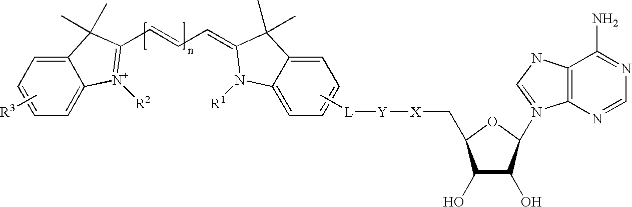
wherein,
n is 1, 2 or 3;
R¹, R², and R³ are independently selected from optionally substituted alkyl, alkenyl, aryl, alkylaryl and heteroatom-inserted alkyl groups;
X is —CH₂, —CO—, —O—, —S—, —NH—, —NR—, —ONH—, or —CONH—;
Y is —CH₂—, —CO—, —PO₃—, P(S)O₂—, —SO₂—, —COO—, —CO—, —PO₂NH, S or O; and
L is a linker of at least 2 and up to about 50 atoms in length optionally substituted with one or more heteroatoms.

3. The adenosine derivative of claim 2, wherein R¹ and R² are the same.

4. The adenosine derivative of claim 3, wherein R¹ and R² are methyl, ethyl, propyl and hydroxypropyl group.

5. The adenosine derivative of claim 2, having the following structure:

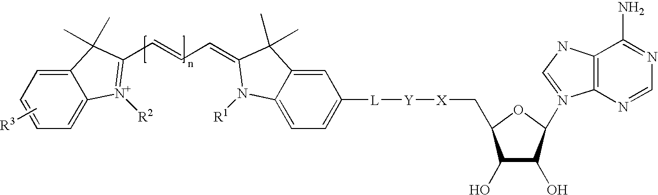

6. The adenosine derivative of claim 5, having the following structure: 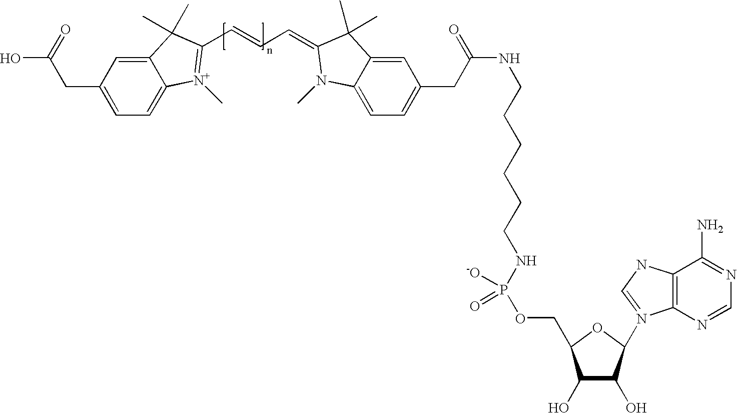
wherein n is 1, 2 or 3.

7. An adenosine derivative having two adenosine moieties covalently linked to a symmetric, fluorescent cyanine dye through the 5′ positions of each adenosine residue.

8. The adenosine derivative of claim 7, having the following structure: 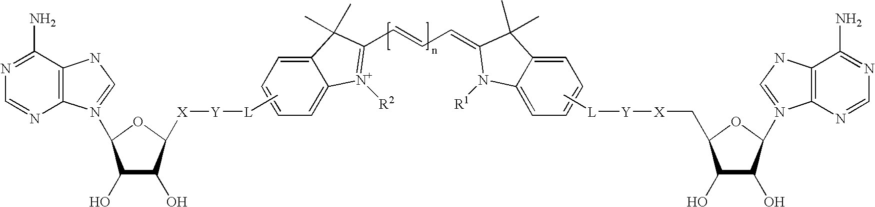
wherein,
n is 1, 2 or 3;
R¹, R² are independently selected from optionally substituted alkyl, alkenyl, aryl, alkylaryl, and heteroatom-inserted alkyl groups;
X is —CH₂, —CO—, —O—, —S—, —NH—, —NR—, —ONH—, or —CONH—;
Y is —CH₂—, —CO—, —PO₃—, P(S)O₂—, —SO₂—, —COO—, —CO—, —PO₂NH, S or O; and
L is a linker of at least about 2 and up to about 50 atoms in length optionally substituted with one or more heteroatoms.

9. The adenosine derivative of claim 8, having the following structure:

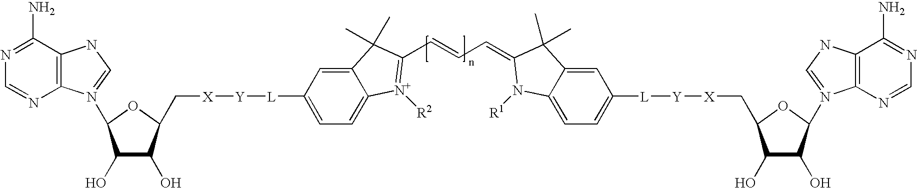

10. The adenosine derivative of claim 9, wherein X is O.

11. The adenosine derivative of claim 10, wherein Y is —P(═O)O³¹—.

12. The adenosine derivative of claim 9, wherein R¹ and R² are the same.

13. The adenosine derivative of claim 9, wherein R¹ and R² are methyl, ethyl, propyl or hydroxypropyl groups.

14. The adenosine derivative of claim 11, having the structure: 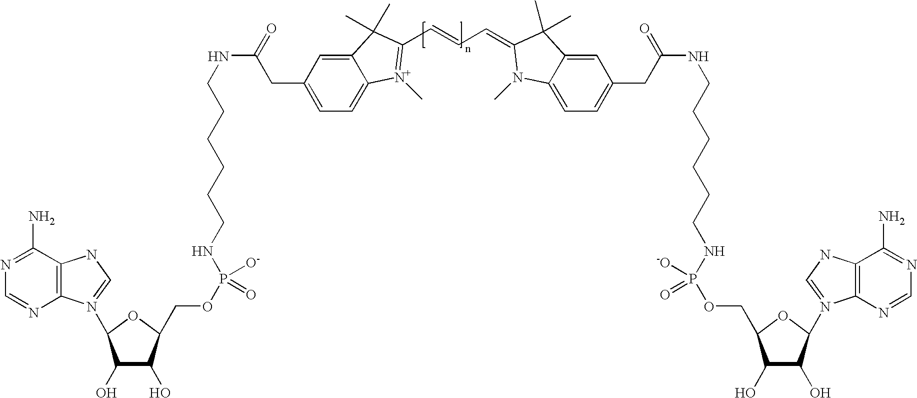
wherein n is 1, 2 or 3.

15. A method for producing labeled RNA, the method comprising:
providing a sample having a first nucleic acid;
amplifying a nucleic acid sequence in the first nucleic acid with a first and a second primer, wherein the first primer contains an RNA polymerase promoter sequence, to produce an amplicon;
transcribing the amplicon in the presence of:
an RNA polymerase that specifically recognizes the RNA polymerase promoter sequence; and
a 5′-labeled adenosine derivative.

16. The method of claim 15, wherein amplifying is carried out using the polymerase chain reaction.

17. The method of claim 15, wherein the promoter sequence is a T7 class II promoter sequence.

18. The method of claim 17, wherein the polymerase is a T7 RNA polymerase.

19. The method of claim 15, wherein the adenosine derivative is labeled at the 5′ position with an electrochemical tag, a metal complex, an affinity tag, a co-enzyme, or a fluorescent dye.

20. The method of claim 19, wherein the affinity tag is biotin or a biotin derivative.

21. The method of claim 19, wherein the labeled adenosine is an adenosine derivative of claim 1.

22. The method of claim 19, wherein the labeled adenosine is an adenosine derivative of claim 7.
